(19) United States
(12) Patent Application Publication
(10) Pub. No.: US 2006/0130173 A1
(43) Pub. Date: Jun. 15, 2006

(54) Genetic modification of plants for enhanced resistance and decreased uptake of heavy metals

(76) Inventors: Youngsook Lee, Pohang-city (KR); Young-Yell Yang, Pohang-city (KR); Inhwan Hwang, Pohang-city (KR); Hyunjoo Bae, Daegu-city (KR); Joohyun Lee, Seoul (KR); Enrico Martinoia, Zurich (CH)

Correspondence Address: JHK Law, P.O. Box 1078, La Canada, CA, 91012-1078, US

(21) Appl. No.: 10/756,047

(22) Filed: Jan. 12, 2004

Related U.S. Application Data
(63) Continuation-in-part of application No. 10/474,290, filed on Oct. 1, 2003.
(60) Provisional application No. 60/526,161, filed on Dec. 2, 2003.

(51) Int. Cl. A01H 1/00; C12N 9/22; C12N 15/82; C07K 14/415

(52) U.S. Cl. 800/278; 435/199; 435/468; 435/419; 530/370

(57) ABSTRACT

The present invention relates to a method of producing transformants with enhanced resistance and decreased uptake of heavy metals, and a plant transformed with a P type ATPase ZntA gene that pumps out heavy metals from the cells. The transformants show better growth than wild type in environment contaminated with heavy metals and have lower heavy metal contents than wild type plants. Therefore, this method of transforming plants with ZntA or biologically active ZntA-like heavy metal pumping ATPases can be useful for developing plants for phytoremediation and also for a safe crop that has resistance to heavy metals and low heavy metal contents.

### BACKGROUND OF THE INVENTION

1. Field of the Invention

The present invention relates to a method of producing transformants with enhanced heavy metal resistance. More particularly, the present invention relates to transgenic plants that have an improved growth but decreased heavy metal content when grown in environment contaminated with heavy metals, thus this method can be used for developing plants for phytoremediation and also for developing safe crops.

2. Description of the Related Art

Heavy metals are major environmental toxicants, which cause reactive oxidation species generation, DNA damage, and enzyme inactivation by binding to active sites of enzymes in cells.

Contamination of the environment with heavy metals has increased drastically due to industrialization. By the early 1990s, the worldwide annual release had reached 22,000 tons of cadmium, 954,000 tons of copper, 796,000 tons of lead, and 1,372,000 tons of zinc (Alloway B J & Ayres D C (1993) Principles of environmental pollution. Chapman and Hall, London). The soils contaminated with heavy metal inhibit normal plant growth and cause contamination of foodstuffs. Many heavy metals are very toxic to human health and carcinogenic at low concentrations. Therefore removal of heavy metals from the environment is an urgent issue.

Studies for removing heavy metals from soil are very actively progressing worldwide. Traditional methods of dealing with soil contaminants include physical and chemical approaches, such as the removal and burial of the contaminated soil, isolation of the contaminated area, fixation (chemical processing of the soil to immobilize the metals), and leaching using an acid or alkali solution (Salt D E, Blaylock M, Kumar NPBA, Viatcheslav D, Ensley B D, et al. (1995). Phytoremediation: a novel strategy for the removal of toxic metals from the environment using plants. Bio-Technology 13,468-74; Raskin I, Smith R D, Salt D E. (1997) Phytoremediation of metals: using plants to remove pollutants from the environment. Curr Opin. Biotechnol. 8, 221-6). These methods, however, are costly and energy-intensive processes.

Phytoremediation has recently been proposed as a low-cost, environment-friendly way to remove heavy metals from contaminated soils, and is a relatively new technology for cleanup of contaminated soil that uses general plants, specially bred plants, or transgenic plants to accumulate, remove, or detoxify environmental contaminants. The phytoremediation technology is divided into phytoextraction, rhizofiltration, and phytostabilization.

Phytoextraction is a method using metal-accumulating plants to extract metals from soil into the harvestable parts of the plants; rhizofiltration is a method using plant roots to remove contaminants from polluted aqueous streams; and phytostabilization is the stabilization of contaminants such as toxic metals in soils to prevent their entry into ground water, also with plants (Salt et al., Biotechnology 13(5): 468-474, 1995).

Examples of phytoremediation are methods using the plants of Larrea tridentate species that are particularly directed at the decontamination of soils containing copper, nickel, and cadmium (U.S. Pat. No. 5,927,005), and a method using Brassicaceae family (Baker et al., New Phytol. 127:61-68, 1994).

In addition, phytoremediation using transgenic plants that are generated by introducing genes having resistant activity for heavy metals have been attempted. Examples of heavy metal resistant genes are CAX2 (Calcium exchanger 2), cytochromium P450 2E1, NtCBP4 (Nicotiana tabacum calmodulin-binding protein), GSHII (glutathione synthetase), merB (organomercurial lyase), and MRT polypeptide (metal-regulated transporter polypeptide) examples of which are IRT1 and IRT2 (iron-regulated transporter).

CAX2 (Calcium exchanger 2), isolated from Arabidopsis thaliana, accumulates heavy metals including cadmium and manganese in plants (Hirschi et al., Plant Physiol. 124:125-134, 2000). Cytochromium P450 2E1 uptakes and decomposes organic compounds such as trichloroethylene (Doty S L et al., Proc. Natl. Acad. Sci. USA 97:6287-6291, 2000). Nicotiana tabacum transformed with NtCBP4 has resistant activity for nickel (Arazi et al., Plant J. 20:171-182, 1999), GSHII accumulates cadmium (Liang et al., Plant Physiol. 119:73-80, 1999), merB detoxifies organic mercury (Bizily et al., Proc. Natl. Acad. Sci. USA 96:6808-6813, 1999), and MRT polypeptide removes heavy metals including cadmium, zinc, and manganese from contaminated soil (U.S. Pat. No. 5,846,821).

However, the transgenic plants generated by introducing the above-mentioned genes have limitations in growth due to accumulation of heavy metals, and they can produce contaminated fruits and crops, when grown in contaminated soil. Therefore, there is a need for plants that have a lower uptake of heavy metals than the wild type, and that maintain healthy growth even in an environment contaminated with heavy metals.

### SUMMARY OF THE INVENTION

In one aspect, the invention is directed to a gene, when expressed in plants, that confers heavy metal resistance and that can inhibit accumulation of heavy metals.

In another aspect, the invention provides a recombinant vector harboring a heavy metal resistant gene.

In still another aspect, the invention provides a method for producing transformants that have heavy metal resistance and that accumulate less heavy metals than wild type plants.

In another aspect, the invention provides transformants that have heavy metal resistance and that accumulate less heavy metals than wild type plants.

In yet another aspect, the invention provides a method of transforming a polluted area into an environmentally friendly space.

The invention provides a recombinant vector containing a coding sequence for a heavy metal-transporting P type ATPase, wherein the coding sequence is operably linked to and under the regulatory control of a plant-expressible transcription and translation regulatory sequence.

Also, the invention provides a transgenic plant, or parts thereof, each transformed with a recombinant vector.

Also, the invention provides a transgenic plant cell.

Also, the invention provides a transgenic plant, stably transformed with a recombinant vector.

Also, the invention provides a recombinant vector comprising a coding sequence for a heavy metal-transporting P type ATPase. In particular, the gene is ZntA. Further in particular, the gene may have a sequence that is at least about 50% similar to SEQ ID NO: 1;

wherein the coding sequence is operably linked to and under the regulatory control of a plant-expressible transcription and translation regulatory sequence; and

wherein the ZntA contains an approximately 100 amino acid residue N-terminal extension domain, a first transmembrane spanning domain, a second transmembrane spanning domain containing a putative cation channel motif CPX domain, a third transmembrane spanning domain, a first cytoplasmic domain, a second cytoplasmic domain, and a C-terminal domain

Also, the invention provides a recombinant vector comprising a coding sequence for a heavy metal-transporting P type ATPase, ZntA wherein the coding sequence is operably linked to and under the regulatory control of a plant-expressible transcription and translation regulatory;

wherein the ZntA contains an approximately 100 amino acid residue N-terminal extension domain, a first transmembrane spanning domain, a second transmembrane spanning domain containing a putative cation channel motif CPX domain, a third transmembrane spanning domain, a first cytoplasmic domain, a second cytoplasmic domain, and a C-terminal domain; and

wherein each of the domains of the coding sequence shares at least about 50% homology with a same domain of SEQ ID NO: 1.

Also, the invention provides a method of producing a transgenic plant with enhanced resistance to heavy metals comprising:

(a) preparing an expression construct comprising a sequence encoding a heavy metal-transporting P type ATPase, operably linked to and under the regulatory control of a plant-expressible transcription and translation regulatory sequence;

(b) preparing a recombinant vector harboring the expression construct; and

(c) introducing the expression construct of the recombinant vector into a plant cell or plant tissue to produce a transgenic plant cell or transgenic plant tissue.

The present invention is directed in particular to transgenic tobacco plants comprising ZntA-like gene. The obtained tobacco leaves are low in cadmium content, which reduces health risk for tobacco smokers.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings, which are given by way of illustration only, and thus are not limitative of the present invention, and wherein;

FIG. 1 represents the map of the recombinant vector pEZG.

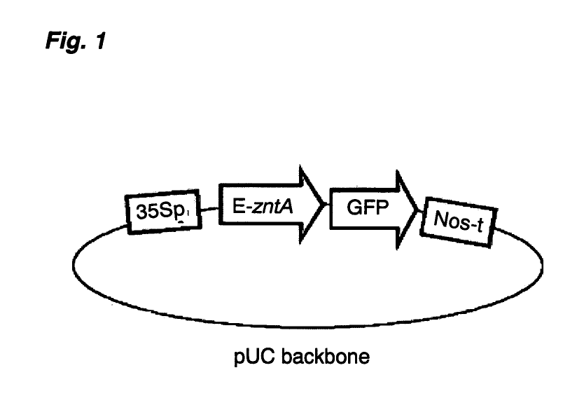

FIG. 2 shows plasma membrane localization of ZntA protein expressed in Arabidopsis protoplasts.

FIG. 3 is a Western blot photograph showing membrane localization of ZntA protein expressed in Arabidopsis protoplast.

FIG. 4 represents the map of recombinant vector PBI121/zntA.

FIG. 5 is a Northern blot photograph showing expression of znLA mRNA in Arabidopsis.

FIG. 6 shows the enhanced growth of zntA-transgenic plants over that of wild type in a medium containing lead.

FIG. 7 shows the enhanced growth of zntA-transgenic plants over that of wild type in a medium containing cadmium.

FIG. 8 is a graph showing the weight of zntA-transgenic plants cultivated in media containing heavy metals.

FIG. 9 is a graph showing the chlorophyll contents of zntA-transgenic and wild type plants, grown in media containing heavy metals.

FIG. 10 is a graph showing the heavy metal contents of zntA-transgenic and wild type plants, grown in media containing heavy metals.

FIG. 11 shows PCR-amplification of znLA gene from the plants transformed. Genomic DNA extracted from individual lines of transgenic tobacco plants and wild type tobacco plants was used as template, and the primers were taken from 35S promoter region and zntA gene. The expected size of the product amplified from the inserted gene is 2.5 kb.

### DETAILED DESCRIPTION OF THE PRESENT INVENTION

In the present application, “a” and “an” are used to refer to both single and a plurality of objects.

As used herein, the term “P type ATPase” refers to a transporter that transports a specific material by using energy from ATP hydrolysis and that forms a phosphorylated intermediate. More particularly, the P type ATPase is a heavy metal-transporting ATPase. The heavy metal is a metal element having a specific gravity over 4 including arsenic(As), antimony(Sb), lead(Pb), mercury(Hg), cadmium(Cd), chromium(Cr), tin(Sn), zinc, barium(Ba), nickel(Ni), bismuth(Bi), cobalt(Co), manganese(Mn), iron(Fe), copper(Cu), and vanadium(V).

ZntA is a P type ATPase of E. coli (Rensing C, Mitra B, Rosen B P. (1997) Proc. Natl. Acad. Sci. U S A. 94,14326-31; Sharma, R., Rensing, C., Rosen, B. P., Mitra, B. (2000) J Biol Chem. 275,3873-8) which pumps Pb(II)/Cd(II)/Zn(II) across the plasma membrane.

P-type ATPases typically have 2 large cytoplasmic domains and 6 transmembrane domains. ZntA has similar domains, and in addition, 2 more transmembrane helixes at N-terminus and N-terminal extension of about 100 amino acids containing CXXC motif. The first large cytoplasmic domain of ZntA is about 145 amino acid long and involved in hydrolysis of phosphointermediate, and the second large cytoplasmic domain is 280 amino acid long and has a phosphorylation motif. We denote the 4 transmembrane helixes of the N-terminal side as the first transmembrane spanning domain. The 2 transmembrane helixes between the 2 large cytoplasmic domains is denoted as the second transmembrane spanning domain. This domain contains a putative cation channel motif CPX domain. The transmembrane helixes between the second large cytoplasmic domain and the c-terminus is denoted as the third transmembrane spanning domain. The cytoplasmic domain following the third transmembrane spanning domain is denoted as the C-terminal domain of ZntA.

The term “homology” refers to the sequence similarity between 2 DNA or protein molecules. “Biologically active ZntA-like heavy metal pumping ATPases” are coded by DNA sequences which have at least 50% homology to ZntA, and have heavy metal pumping activity. Biologically active ZntA-like heavy metal pumping ATPases include zinc-transporting ATPase (NC_{—}000913), zinc-transporting ATPase (NC_{—}002655), heavy metal-transporting ATPase (NC_{—}003198), P-type ATPase family (NC_{—}003197), cation transporting P-type ATPase from Mycobacterium lepraed (GenBank #Z46257), and many others.

As used herein, the term “sequence identity”, “sequence similarity” or “sequence homology” means nucleic acid or amino acid sequence identity in two or more aligned sequences, aligned using a sequence alignment program. Sequence searches are preferably carried out using the BLASTN program when evaluating the of a given nucleic acid sequence relative to nucleic acid sequences in the GenBank DNA Sequences and other public databases. The BLASTX program is preferred for searching nucleic acid sequences which have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62 matrix. (See, Altschul, et al., 1997.)

The term “% homology” is used interchangeably herein with the term “% identity” herein and refers to the level of identity between two sequences, i.e. 70% homology means the same thing as 70% sequence identity as determined by a defined algorithm, and accordingly a homologue of a given sequence has at least about 70%, preferably about 80%, more preferably about 85%, even more preferably about 90% sequence identity over a length of the given sequence.

A preferred alignment of selected sequences in order to determine “% identity” between two or more sequences, is performed using the CLUSTAL-W program in MacVector version 6.5, operated with default parameters, including an open gap penalty of 10.0, an extended gap penalty of 0.1, and a BLOSUM 30 similarity matrix.

A “heavy metal resistance protein” is a protein capable of mediating resistance to at least one heavy metal, including, but not limited to, lead, cadmium, and zinc. An example of a heavy metal resistance protein is ZntA protein of SEQ ID NO:1.

The term “plant-expressible” means that the coding sequence is operably linked to and under the regulatory control of a transcription and translation regulatory sequence that can be efficiently expressed by plant cells, tissues, parts and whole plants.

“Plant-expressible transcriptional and translational regulatory sequences” are those which can function in plants, plant tissues, plant parts and plant cells to effect the transcriptional and translational expression of the target sequence with which they are associated. Included are 5′ sequences of a target sequence to be expressed, which qualitatively control gene expression (turn gene expression on or off in response to environmental signals such as light, or in a tissue-specific manner); and quantitative regulatory sequences which advantageously increase the level of downstream gene expression. An example of a sequence motif that serves as a translational control sequence is that of the ribosome binding site sequence. Polyadenylation signals are examples of transcription regulatory sequences positioned downstream of a target sequence, and there are several that are well known in the art of plant molecular biology.

A “transgenic plant” is one that has been genetically modified, unlike the wild type plants. Transgenic plants typically express heterologous DNA sequences, which confer the plants with characters different from that of wild type plants. As specifically exemplified herein, a transgenic plant is genetically modified to contain and express at least one heterologous DNA sequence that is operably linked to and under the regulatory control of transcriptional control sequences which function in plant cells or tissue, or in whole plants.

The present invention provides a plant-expressible expression construct containing a coding sequence for a heavy metal-transporting ATPase protein. The coding sequence is operably linked to and under the regulatory control of a plant-expressible transcription and translation regulatory sequence. The heavy metals include arsenic(As), antimony(Sb), lead(Pb), mercury(Hg), cadmium(Cd), chromium(Cr), tin(Sn), zinc, barium(Ba), nickel(Ni), bismuth(Bi), cobalt(Co), manganese(Mn), iron(Fe), copper(Cu) and vanadium(V).

The expression construct includes a promoter, a heavy metal-transporting P type ATPase gene, and a transcriptional terminator. The suitable plant-expressible promoters include the 35S or 19S promoters of Cauliflower Mosaic Virus; the nos (nopaline synthase), ocs (octopine synthase), or mas (mannopine synthase) promoters of Agrobacterium tumefaciens Ti plasmids; and others known to the art.

The heavy metal-transporting ATPase gene of the present invention prefers genes encoding ZntA (SEQ ID NO:1) or biologically active ZntA-like heavy metal pumping ATPase genes, which have at least 50%, 70%, 80%, 90%, 95%, 97%, 99% or 100% homology to ZntA, and which code for proteins with heavy metal pumping activities.

The heavy metal-transporting ATPase gene of the present invention also prefers DNA sequences containing an approximately 100 amino acid residue N-terminal extension domain, a first transmembrane spanning domain, a second transmembrane spanning domain containing a putative cation channel motif CPX domain, a third transmembrane spanning domain, a first cytoplasmic domain, a second cytoplasmic domain, and a C-terminal domain of ZntA, or DNA sequences which share at least 50%, 70%, 80%, 90%, 95%, 97%, 99% or 100% homology with abovementioned domains of the biologically active ZntA-like heavy metal pumping ATPase genes.

The expression construct of the present invention may further contain a marker allowing selection of transformants in the plant cell or showing a localization of a target protein. The examples of a marker without limitation are genes carrying resistance to an antibiotic such as kanamycin, hygromycin, gentamycin, and bleomycin; and genes coding GUS (β-glucuronidase), CAT (chloramphenicol acetyltransferase), luciferase, and GFP (green fluorescent protein). The marker allows for selection of successfully transformed plant cells growing in a medium containing certain antibiotics because they will carry the expression construct with the resistance gene to the antibiotic.

Also, the invention provides a recombinant vector comprising the expression construct. The recombinant vector comprises a backbone of the common vector and the expression construct. The common vector is preferably selected from the group consisting of pROKII, pBI76, pET21, pSK(+), pLSAGPT, pBI121, and pGEM. Examples of the prepared recombinant vector are PBI121/zntA and pEZG. PBI121/zntA comprises a backbone of PBI121, CMV 35S promoter, zntA gene, and nopaline synthase terminator; and pEZG comprises a backbone of pUC, CMV 35S promoter, znLA gene, green fluorescence protein, and nopaline synthase terminator.

Also, the present invention provides a transformant containing the expression construct. The transformant contains a DNA sequence encoding a heavy metal-transporting P type ATPase, wherein the coding sequence is operably linked to and under the regulatory control of a transcription and translation regulatory sequence.

The transformant is preferably a plant, and more preferably a plant part thereof, or plant cell. The plant part includes a seed. The plants may be without limitation herbaceous plants and trees, and may include flowering plants, garden plants, an onion, a carrot, a cucumber, an olive tree, a sweet potato, a potato, a cabbage, a radish, lettuce, broccoli, tobacco such as Nicotiana tabacum, Petunia hybrida, a sunflower, Brassica juncea, turf, Arabidopsis thaliana, Brassica campestris, Betula platyphylla, a poplar, a hybrid poplar, or Betula schmidtii.

Techniques for generating transformants are well known. An example is Agrobacterium tumefaciens-mediated DNA transfer. Preferably, recombinant A. tumefaciens generated by electroporation, micro-particle injection, or with a gene gun can be used.

In addition, the invention provides a method of producing a transgenic plant with enhanced resistance to heavy metals, comprising:

(a) preparing an expression construct comprising a plant-expressible sequence encoding a heavy metal-transporting P type ATPase, operably linked to and under the regulatory control of a transcription and translation regulatory sequence;

(b) preparing a recombinant vector harboring the expression construct; and

(c) introducing the expression construct of the recombinant vector into a plant cell or plant tissue to produce a transgenic plant cell or transgenic plant tissue.

The method of producing a transgenic plant further comprises a step: (d) regenerating a transgenic plant from the transgenic plant cell or transgenic plant tissue of step (c).

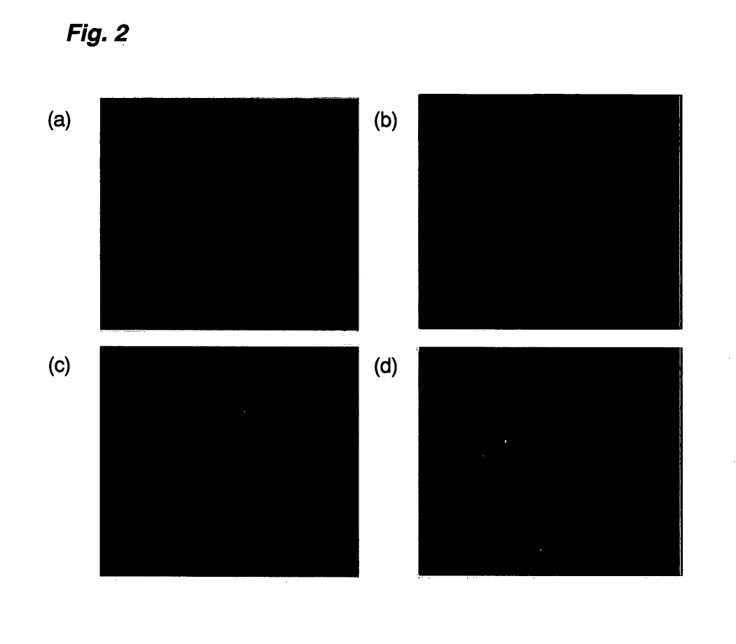

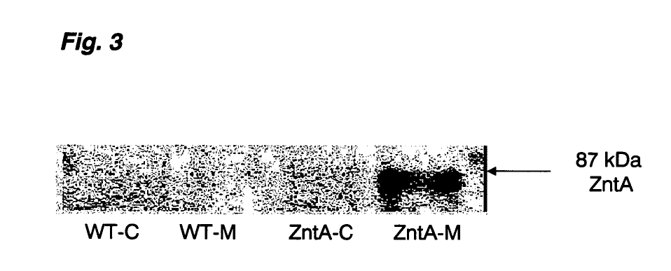

In the present invention, ZntA protein was expressed in the plasma membrane (FIGS. 2 and 3). Moreover, zntA-transgenic Arabidopsis plants showed enhanced resistance to lead and cadmium, and the content of lead and cadmium was lower than in a wild-type plant.

Therefore, zntA-transgenic plants or plants transformed with a gene encoding biologically active ZntA-like heavy metal pumping ATPases can grow in an environment contaminated with heavy metals, and this technique can be useful for generating crop plants with decreased uptake of harmful heavy metals. Since harmful heavy metals can be introduced into farmland inadvertently, for example, due to the yellow sand phenomenon or by natural disaster, heavy metal pumping transgenic crop plants can be a safe choice for health-concerned consumers.

Tobacco

Tobacco is a tall leafy annual plant that belongs to the solanaceae or nightshade family, which consists of crop plants, perennial flowering plants, poisonous weeds, various herbs, shrubs and trees. Tobacco includes numerous species, which are grown throughout the world. One species, Nicotiana tabacum or common tobacco, is the main source of commercial tobacco used in producing cigarettes. It is native to South America, Mexico and the West Indies. Most species of tobacco are believed to be native to the Western Hemisphere.

Much of the tobacco grown in the United States is common_tobacco. Two varieties of large-leaf tobacco are bright-leaf and burley tobacco. About 50% of the tobacco grown in the United States is bright-leaf tobacco and approximately 40% is burley tobacco. These typically large-leaf varieties can grow to reach 1.2 to 1.8 meters (4 to 6 feet in height). Each plant produces approximately 20 leaves measuring from 60-75 centimeters (24-30 inches) long and 38-46 centimeters (15-18 inches) wide. The colors vary from yellow to green including all shades in between. Most are covered with hairs that secrete a thick and sticky liquid. As the plant matures, an irregularly branched flower cluster develops at its top. These flowers are either white or pink.

Each variety of tobacco has different growing requirements. All of these requirements must be met to produce a healthy crop. Tobacco grows in many types of soil (including silt and silt-loam, sandy and sandy-loam, and clay and clay-loam soil) and in varying climates (requiring a frost free period of 100-130 days). Differences in soil and climate produce leaves that have specific characteristics and require different methods of fertilization, insect and disease management, harvesting and curing.

The invention is directed to zntA-transgenic tobacco showing enhanced resistance to cadmium, wherein the content of cadmium in the transgenic tobacco plant is lower than the wild-type tobacco plant.

The following examples are provided for illustrative purposes and are not intended to limit the scope of the invention as claimed herein. Any variations in the exemplified compositions and methods which occur to the skilled artisan are intended to fall within the scope of the present invention.

### EXAMPLE 1

Isolation of zntA Gene

Escherichia coli K-12 was obtained from the Korean Collection for Type Cultures of the Korea Research Institute of Bioscience and Biotechnology, and a znta gene was cloned.

zntA was isolated by PCR using genomic DNA of Escherichia coli K-12 strain as a template. PCR was performed with a primer set of SEQ ID NO:2, SEQ ID NO:3, and 2.2 kb of PCR product, and zntA of SEQ ID NO:1 was obtained. The sequence of the PCR product was analyzed and the PCR product was cloned into a pGEM-T easy vector to produce pGEM-T/zntA.

### EXAMPLE 2

Expression of ZntA Protein

A zntA gene was introduced into Arabidopsis protoplasts, and localization of ZntA protein was investigated.

(2-1) Preparation of Arabidopsis Protoplasts

Arabidopsis protoplasts were prepared as described (Abel S, Theologis A (1994) Transient transformation of Arabidopsis leaf protoplasts: a versatile experimental system to study gene expression. Plant J. 5, 421-7).

Seeds of Arabidopsis were placed into an antiseptic solution (distilled water: chlorox: 0.05% triton X-100=3:2:2), shaken for 20-30 seconds, and incubated at room temperature for 5-10 mins. The seeds were then rinsed five times with distilled water.

The Arabidopsis seeds were incubated in 100 ml of a liquid solution (Murashige & Skoog medium; MSMO, pH 5.7-5.8) containing vitamins, Duchefa 4.4 g/L, sucrose 20 g/L, MES (2-(N-Morpholino) Ethanesulfonic acid, Sigma) 0.5 g/L, while agitating at 120 rpm under a 16/8 hr (light/dark) cycle, at 22° C. for 2-3 weeks.

The 2-3 week-old whole plants were chopped with a razor blade to 5-10 mm² pieces. These leaf fragments were transferred to an enzyme solution (1% cellulase R-10, 0.25% marcerozyme R-10, 0.5 M mannitol, 10 mM MES, 1 mM CaCI₂, 5 mM β-mercaptoethanol, and 0.1% BSA, pH 5.7-5.8), vacuum-infiltrated for 10 min, and then incubated in the dark at 22° C. for 5 hours with gentle agitation at 50-75 rpm. The released protoplasts were filtered through a 100 μm mesh (Sigma S0770, USA), purified using a 21% sucrose gradient by centrifugation at 730 rpm for 10 min, and then suspended in 20 ml of W5 solution (154 mm NaCl, 125 mM CaCl₂, 5 mM KCl, 5 mM glucose, and 1.5 mM MES, pH 5.6) and centrifuged again at 530 rpm for 6 min. The pellected protoplasts were re-suspended in W5 solution and kept on ice.

(2-2) Preparation of Vector

pGEM-T/zntA DNA was cut with BamHI restriction enzyme and zntA genes were extracted (QIAGEN Gel extraction kit). The zntA genes were placed under the control of a Cauliflower Mosaic Virus 35S promoter, fused with and then inserted into a pUC-GFP vector containing Green Fluorescent Protein(GFP) and nopaline synthase terminator(NOS), to thereby produce pEZG.

(2-3) Preparation of Vector for H⁺ Pumping Gene

A hydrogen ion pump gene of Arabidopsis, AHA2 cDNA (Gene Bank: P19456), was amplified by PCR. Primers for PCR were polynucleotides of SEQ ID NO:4 and SEQ ID NO:5. PCR conditions were as follows: 94° C., 30 sec→45° C., 30 sec→72° C., 1 min, 50 cycles. The PCR product was obtained as AHA2 cDNA.

A DsRed vector (Clontech, Inc.) was treated with BglII/NotI restriction enzyme and DsRed was obtained. The DsRed was inserted into the opened smGFP vector with a BamHI/Ecl136II restriction enzyme to 326RFP. In addition, AHA2 cDNA was inserted at XmaI of the 326RFP vector and 326RFP/AHA2 was prepared.

(2-4) Introduction of pEZG or 326RFP/AHA2 into Protoplast

pEZG and 326RFP/AHA2 were introduced to the protoplasts prepared by EXAMPLE (2-1), and expression of foreign genes was confirmed.

The protoplast was centrifuged at 500 rpm for 5 min, and 5×10⁶/ml of the protoplast were suspended in a MaMg solution (400 mM mannitol, 15 mM MgCl₂, 5 mM MES-KOH, pH 5.6). 300 μl of the suspension solution was mixed with 10 μg of pEZG and 326RFP/AHA2 respectively, which was then was added to 300 μl of PEG (400 mM mannitol, 100 mM Ca(NO₃)₂, 40% PEG 6000), and stored at RT for 30 min. The mixture was washed with 5 ml of W5 solution, centrifuged at 500 rpm for 3 min, and a pellet was obtained. The pellet was washed with 2 ml of W5 solution and incubated in the dark at 22-25° C. After 24 hr, expression of GFP protein was monitored and images were captured with a cooled charge-coupled device camera using a Zeiss Axioplan fluorescence microscope. The filter sets used for the GFP were XF116 (exciter, 474AF20; dichroic, 500DRLP; emitter, 510AF23) (Omega, Inc., Brattleboro, Vt.). Data were then processed using Adobe (Mountain View, Calif.) Photoshop software.

FIG. 2 shows a localization of ZntA protein fused with GFP in protoplasts transformed with pEZG and 326RFP/AHA2, respectively. “a” is control, “b” is AHA2 protein expressed in 326RFP/AHA2, “c” is ZntA protein expressed in pEZG, and “d” is an overlapped picture of “b” and “c”. ZntA fused with GFP shows a green color due to GFP, and AHA2 fused with DsRed shows a red color due to DsRed.

In FIG. 2, ZntA fused with GFP was localized at the plasma membrane in Arabidopsis protoplasts.

In addition, membrane and cytosol fractions were isolated from Arabidopsis protoplasts, and Western Blot was preformed using a GFP antibody as a probe. FIG. 3 is a Western Blot photograph, wherein “WT-C” is cytosol of wild-type Arabidopsis protoplasts, “WT-M” is membrane of wild-type Arabidopsis protoplasts, “ZntA-C” is cytosol of Arabidopsis protoplasts transformed with pEZG, and “ZntA-M” is membrane of Arabidopsis protoplasts transformed with pEZG. In FIG. 3, the GFP antibody cross-reacted only with membrane proteins extracted from Arabidopsis protoplasts transformed with pEZG, confirming that ZntA protein was expressed in membrane.

### EXAMPLE 3

Preparation of transgenicArabidopsis plants expressing ZntA protein.

(3-1) Arabidopsis

Arabidopsis plants were grown at 4° C. for 2 days, then they were grown with a 16/8 hr (light/dark) photoperiod, at 22° C./18° C. for 3-4 weeks until flower stalks were differentiated. The 1^{st} flower stalk was removed, and the 2^{nd} flower stalk was used for transformation.

(3-2) pBI121/ zntA Vector

A zntA gene was inserted into the expression vector for the plant, preparing pBI121 and pBI121/zntA.

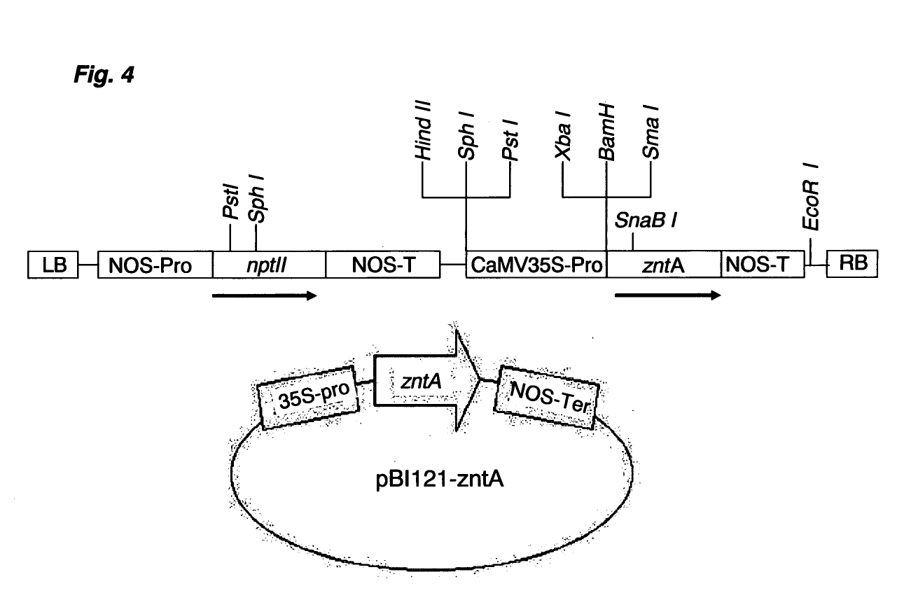

A GUS gene of pBI121 was removed by digesting with Smal and Ecl136II restriction enzymes, and a zntA gene prepared from the pGEM-T/zntA was inserted to pBI121, thereby preparing a pBI121/zntA vector (FIG. 4).

(3-3) Preparation of Transgenic Plants

pBI121/zntA vector DNA was isolated with a prep-kit (Qiagen) and introduced to Agrobacterium using electroporation. The Agrobacterium (KCTC 10270BP) was cultured in YEP media (yeast extract 10 g, NaCl 5 g, pepton 10 g, pH 7.5) until index of O.D. reached 0.8-1.0. The culture solution was centrifuged, cells were collected and suspended in MS media (Murashige & Skoog medium, 4.3 g/L, Duchefa) containing 5% sucrose, and Silwet L-77 (LEHLE SEEDS, USA) was added as a final concentration of 0.01% just before transformation. For plant transformation, pBI121/zntA was introduced into the Agrobacterium LBA4404 strain, which was then used to transform Arabidopsis by a dipping method (Clough S J, and Bent A F (1988), Floral dip: a simplified method for Agrobacterium-mediated transformation of Arabidopsis thaliana. Plant J. 16, 735-743).

### EXAMPLE 4

Selection of Arabidopsis plant Transformants

For selection of plant transformed with zntA genes, plants were grown in solid Murashige-Skoog (MS) medium containing kanamycin (50 mg/l). T2 or T3 generation seeds were used for the tests. Also, a pBI121 vector was introduced to Arabidopsis and transformants (pBI121 plants) were selected. Seeds were obtained from wild-type Arabidopsis, pBI121 plants, and pBI121/zntA plants, respectively.

To test the ZntA expression level, total RNA was isolated from kanamycin-selected T2 plants and used for Northern Blot analysis. Total RNA was extracted from Arabidopsis plants grown on the 1/2 MS (Murashige & Skoog medium, 2.15 g/L, Duchefa)-agar media for 3 weeks. Subsequent RNA preparation and northern hybridization followed the established method (Sambrook et al. (2001) Molecular Cloning: A laboratory manual (Third Edition), Cold Spring Harbor Laboratory Press, Cold Spring Harbor, NY) with slight modifications.

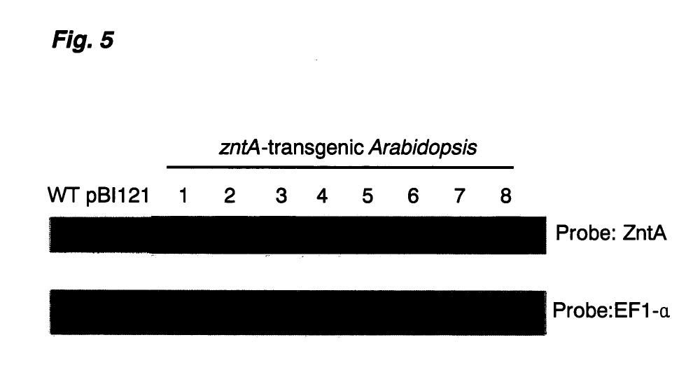

The plant materials were frozen in liquid nitrogen and homogenized with mortars and pestles. 1 ml of TRIzol reagent (Life technology, USA) per 100 mg of tissue was added to the sample and after 5 min incubation at RT, 0.2 ml of chloroform per 1 ml of TRIzol reagent was added. After centrifugation at 10,000 g for 10 min at 4° C., the aqueous phase was taken and precipitated with 0.5 ml of isopropyl alcohol per 1 ml of TRIzol reagent and quantified by UV spectroscopy. Total RNA was separated in a formaldehyde-containing agarose gel and then transferred onto a nylon membrane. After UV crosslinking, hybridization was carried out in a modified Church buffer (7% (w/v) SDS, 0.5 M sodium phosphate (pH 7.2), 1 mM EDTA (pH 7.0)) at 68° C. overnight, with ³²P-labeled zntA probes. Membranes were washed once for 10 min in 1×SSC, 0.1% SDS at room temperature, and twice for 10 min in 0.5×SSC, 0.1% SDS at 68° C. The membrane was exposed to a phosphorimager screen (Fuji film) or x-ray film (Kodak). The mRNA expression levels were analyzed by the Mac-BAS image-reader program. FIG. 5 is a Northern Blot photograph showing expression of znLA mRNA in Arabidopsis. Transcription of zntA RNA was not observed in wild-type Arabidopsis and pBI121 plants, but it was observed in pBI121/zntA plants. EF1-a is constitutively expressed in plants and its even levels indicated that the same amount of RNA was used for different samples.

### EXAMPLE 5

Heavy Metals Resistance of Arabidopsis Transformed with zntA Gene

Wild-type Arabidopsis plants and pBI121/zntA plants were grown in 1/2 MS-agar media for 2 weeks and transferred 1/2 MS-liquid media containing 70 μM cadmium or 0.7 mM lead. After 2 weeks, growth, weight, and heavy metal contents were measured.

(5-1) Growth of Plants

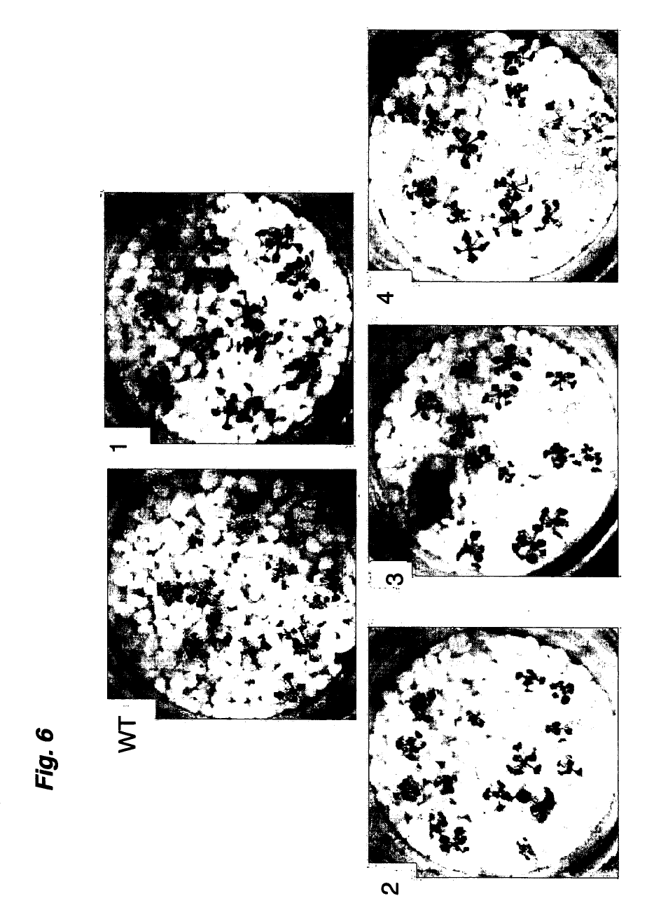

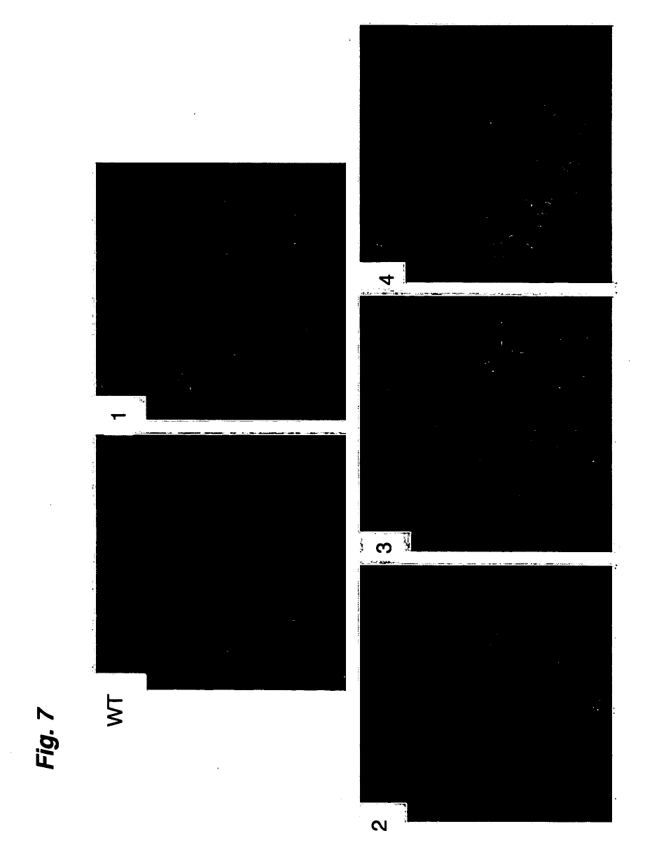

FIG. 6 shows the growth of wild-type and pBI121/zntA Arabidopsis plants grown in a medium containing lead. FIG. 7 shows wild-type and pBI121/zntA Arabidopsis plants grown in a medium containing cadmium. “WT” is wild-type Arabidopsis, “1” to “4” are pBI121/zntA plants. In FIGS. 6 and 7, pBI121/zntA plants grew better than the wild-type plants; their leaves were broader, greener, and their fresh weights were higher than those of the wild types. These results indicate that the expression of ZntA confers Pb(II)- and Cd(II)-resistance to the transgenic plants.

(5-2) Measurement of Biomass

Wild type and pBI121/zntA Arabidopsis plants were grown in 1/2 MS-agar media for 2 weeks and then transferred to 1/2 MS-liquid media supported by small gravel with or without Cd (II) or Pb (II). After growing for an additional 2 weeks, the plants were harvested. They were washed in an ice-cold 1 mM tartarate solution and blot-dried. The weight of the wild type and pBI121/zntA Arabidopsis plants were measured.

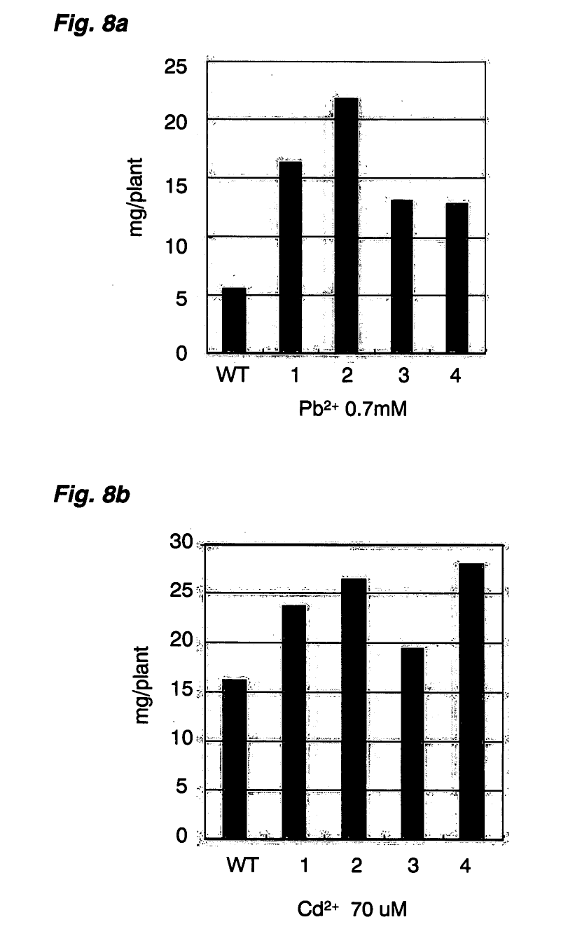

FIG. 8a is a graph showing the weight of wild type and pBI121/zntA plants grown in a medium containing lead, and FIG. 8b is a graph showing the weight of wild type and pBI121/zntA plants grown in a medium containing cadmium. The weight of pBI121/zntA plants was higher than that of the wild-type plants. These results indicate that plants expressing ZntA protein can grow better than wild type in soil contaminated with heavy metals.

(5-3) Measurement of Chlorophyll Contents

For determination of chlorophyll contents, the leaves were harvested and extracted with 95% ethanol for 20 min at 80° C. Absorbance at 664 run and 648 nm were measured and then the chlorophyll A and B contents were calculated as described (Oh S A, Park J H, Lee G I, Paek K H, Park S K, Nam H G (1997) Identification of three genetic loci controlling leaf senescence in Arabidopsis thaliana. Plant J. 12, 527-35).

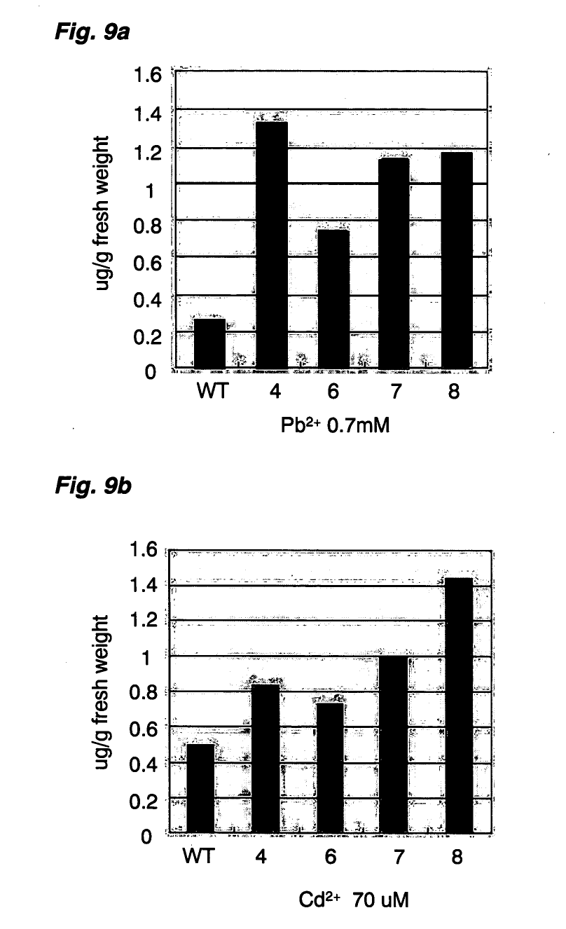

FIG. 9a is a graph showing the chlorophyll contents of wild type and zntA-transgenic plants grown in a medium containing lead, and FIG. 9b is a graph showing the chlorophyll contents of wild type and zntA-transgenic plants grown in a medium containing cadmium. The chlorophyll contents of zntA-transgenic plants were higher than those of the wild types.

(5-4) Measurement of the Heavy Metal Contents

We measured the content of Pb and Cd in control and ZntA overexpressing plants grown in media containing heavy metals. pBI121/zntA plants were collected, weighed, and digested with 65% HNO₃ at 200° C., overnight. Digested samples were diluted with 0.5 N HNO₃ and analyzed using an atomic absorption spectrometer (AAS; SpectrAA-800, Varian).

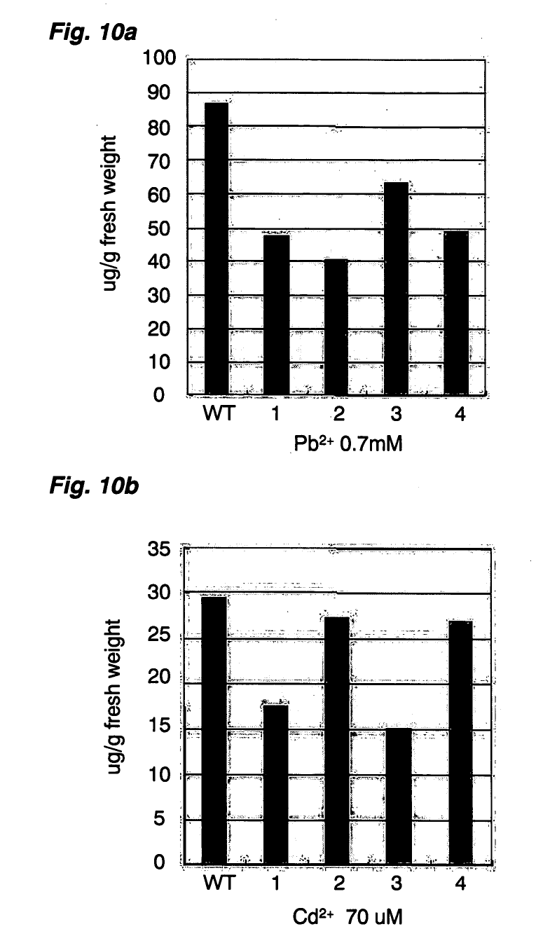

FIG. 10 is a graph showing the heavy metal contents of wild type and zntA-transgenic plants grown in media containing heavy metals. FIG. 10a is the lead contents, and 10b is the cadmium contents. Pb content of pBI121/zntA plants varied between the lines, but it was consistently lower than that of the wild type. Cd content in transgenic lines 1 and 3 was lower than that in the control.

Thus, plants transformed with zntA or other biologically active ZntA-like heavy metal pumping ATPases can be grown in soil contaminated with heavy metals and have less uptake of heavy metals than wild type plants. Since growing plants can hold contaminated soil and thereby reduce erosion of the soil, and since the zntA-transgenic plants can grow better than wild type plants in soil contaminated by heavy metals, they can reduce migration of pollutants from the polluted area, thereby reducing contamination of groundwater by the pollutants. The present invention can also be applied to crop plants to produce low heavy metal -containing safe crop plants.

### EXAMPLE 6

Transformation of Tobacco Leaves with zntA Gene

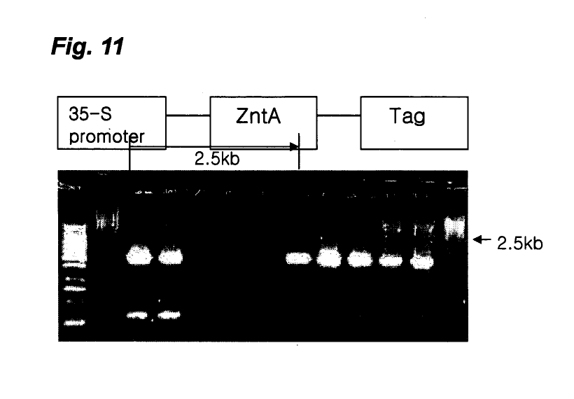

Plant materials: Tobacco (Nicotiana tabaccum L.) seeds were sown and grown at 25° C. for 4 weeks. zntA gene construct introduced into tobacco plants is shown in FIG. 11, and identical to that described in PCT/KR02/00605 (Apr. 04, 2002), Lee et al., Plant Physiology, (in press). Tobacco leaves were cut into approximately 1 cm×1 cm size, rinsed in 70% ethanol and 25% hyperchloride solution to remove microorganisms, and placed onto MS medium containing IBA and NAA. The leaf segments were inoculated with zntA-transformed Agrobacterium and 100 uM Acetosyringone, and incubated in the dark for 3 days at 28° C. Then they were washed to remove Agrobacterium, and incubated in the dark, in MS medium containing 1 mg/L BA, 100 uM Acetosyringone, 250 mg/L cefatoxin, and 200 mg/L kanamycin for 7 days at 28° C., which resulted in development of transgenic calli that are resistant to kanamycin. Shoots were induced by transferring the calli into MS medium containing 1 mg/L BA, 250 mg/L cefatoxin, and 200 mg/L kanamycin for about 1 month at 28° C. Then roots were induced in MS medium containing 250 mg/L cefatoxin and 200 mg/L kanamycin. Plants with more than 3 branches of roots were transplanted to soil and their seeds were collected, dried for 2 weeks, and used for further experiment.

### EXAMPLE 7

Preparation of Contaminated Soil

Common potting soil was mixed with enough CdCl₂ solution to obtain 5 ppm of cadmium. To confirm the contamination level, the soil prepared was heated to 80-85° C., and 0.1 g sample was taken to measure Cd content. The soil was digested in concentrated nitric acid, and the extract was assayed for its cadmium content using atomic absorption spectroscopy. Results from 3 experiments confirmed that we can reliably contaminate the soil to 4-5 ppm using this method.

### EXAMPLE 8

Assay of Cadmium Content of Tobacco Leaves

Wild type and zntA-transgenic tobacco seeds were sown in the same container which contained the soil artificially contaminated with 5 ppm Cd. They were watered once in 2-3 days with tap water. Two months after sowing, 3 wild type plants and one plant each of transformed plants were selected based on their similarity in development; they were all at 7 leaf-stage. The largest leaf from each selected plants was collected for assay of cadmium content. The transformed line 8 did not germinate, and therefore was not tested.

The leaf was rinsed briefly in ice-cold 1 mM tartaric acid, and then with distilled water to remove any contaminant on the surface. It was blot-dried, and its fresh weight was measured. Then the leaf was dried in an oven for 2 days to obtain dry weight. The leaf was then digested in a mixture of HNO₃/HClO₄ solution at 200° C. The volume of the digested sample was adjusted to 10 ml using 0.1 N HNO₃, and its cadmium content was measured using atomic absorption spectroscopy.

### EXAMPLE 9

From a PCR experiment that used genomic DNA extracted from transformed and wild type plants, 8 lines (2, 3, 6, 7, 8, 9, 10, 11) were detected to contain zntA gene (FIG. 11). Lines 1, 4 and 5 as well as the wild type plants did not show the presence of znLA gene.

### EXAMPLE 10

Cadmium Content of Tobacco Leaves

Wild type and zntA-transgenic tobacco plants were grown in the same container, which contained soil artificially contaminated with cadmium at 5 ppm. This level of cadmium is commonly found in agricultural soils in certain European countries, such as France, Spain, Portugal, Czech Republic, and western part of Russia, with Poland exhibiting as much as 1000 ppm cadmium in certain places.

Three replicates of wild type plants and one plant each from 9 transformed plants were assayed for their cadmium contents. Lines 8 and 11 were not included in this test. The transgenic lines 2, 3, 6, 7, 9, 10 that contain zntA gene (FIG. 11) did not contain any detectable level of cadmium in their leaves (Table I). In contrast, 3 wild type plants and the transformed plants that did not show zntA gene (lines 1 and 4) contained cadmium in their leaves. (It is not clear why line 5, which did not show zntA gene had no detectable cadmium. We will repeat this experiment.) Therefore, it can be concluded that by introducing zntA gene into tobacco plants, we successfully reduced the cadmium content of tobacco leaves.

TABLE I
Cadmium content of the wild type and zntA-transgenic tobacco leaves

| | plant | | | | | | | | | | | |
|---|---|---|---|---|---|---|---|---|---|---|---|---|
| | WT (replicates) | | | zntA-transgenic lines | | | | | | | | |
| Menu | 1 | 2 | 3 | 1 | 2 | 3 | 4 | 5 | 6 | 7 | 9 | 10 |
| F.W.^{#} | 75 | 189 | 110 | 122 | 102 | 131 | 178 | 111 | 94 | 81 | 89 | 107 |
| D.W.^{#} | 3.8 | 8.8 | 4.9 | 5.6 | 4.8 | 6.8 | 8.5 | 5.2 | 3.6 | 3.3 | 3.1 | 4.7 |
| Cd | 34 | 22 | 2 | 29 | 0 | 0 | 6 | 0 | 0 | 0 | 0 | 0 |
| content* | | | | | | | | | | | | |

^{#}mg
*mg · kg⁻¹ D.W. of plant

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A recombinant vector comprising a coding sequence for a heavy metal-transporting P type ATPase, wherein the coding sequence is operably linked to and under the regulatory control of a plant-expressible transcription and translation regulatory sequence.

2. The recombinant vector according to claim 1, wherein the heavy metal is at least one selected from the group consisting of arsenic, antimony, lead, mercury, cadmium, chromium, tin, zinc, barium, nickel, bismuth, cobalt, manganese, iron, copper, and vanadium.

3. The recombinant vector according to claim 1, wherein the P type ATPase is ZntA.

4. The recombinant vector according to claim 3, wherein the ZntA has an amino acid sequence, which is at least about 70% similar to SEQ ID NO:2.

5. The recombinant vector according to claim 1, wherein the coding sequence is ZntA-like heavy metal pumping ATPase gene comprising a nucleic acid sequence sharing at least about 70% homology with ZntA as given in SEQ ID NO: 1.

6. The recombinant vector according to claim 1, wherein the recombinant vector is PBI121/zntA or pEZG.

7. A transgenic plant, or parts thereof, transformed with a recombinant vector of claim 1.

8. The transgenic plant, or parts thereof according to claim 7, wherein the heavy metal is at least one selected from the group consisting of arsenic, antimony, lead, mercury, cadmium, chromium, tin, zinc, barium, nickel, bismuth, cobalt, manganese, iron, copper, and vanadium.

9. A transgenic plant cell, transformed with a recombinant vector of claim 1.

10. The transgenic plant cell according to claim 9, wherein the heavy metal is at least one selected from the group consisting of arsenic, antimony, lead, mercury, cadmium, chromium, tin, zinc, barium, nickel, bismuth, cobalt, manganese, iron, copper, and vanadium.

11. A transgenic plant, stably transformed with a recombinant vector of claim 1.

12. The transgenic plant according to claim 11, wherein the heavy metal is at least one selected from the group consisting of arsenic, antimony, lead, mercury, cadmium, chromium, tin, zinc, barium, nickel, bismuth, cobalt, manganese, iron, copper, and vanadium.

13. A transgenic plant, or parts thereof, each transformed with a recombinant vector of claim 5.

14. The transgenic plant, or parts thereof according to claims 13, wherein the heavy metal is at least one selected from the group consisting of arsenic, antimony, lead, mercury, cadmium, chromium, tin, zinc, barium, nickel, bismuth, cobalt, manganese, iron, copper, and vanadium.

15. A transgenic plant cell, transformed with a recombinant vector of claim 5.

16. The transgenic plant cell according to claim 15, wherein the heavy metal is at least one selected from the group consisting of arsenic, antimony, lead, mercury, cadmium, chromium, tin, zinc, barium, nickel, bismuth, cobalt, manganese, iron, copper, and vanadium.

17. A transgenic plant, stably transformed with a recombinant vector of claim 5.

18. The transgenic plant according to claim 17, wherein the heavy metal is at least one selected from the group consisting of arsenic, antimony, lead, mercury, cadmium, chromium, tin, zinc, barium, nickel, bismuth, cobalt, manganese, iron, copper, and vanadium.

19. A recombinant vector comprising a coding sequence for a heavy metal-transporting P type ATPase, ZntA having at least about 70% sequence similarity to SEQ ID NO: 1;
wherein the coding sequence is operably linked to and under the regulatory control of a plant-expressible transcription and translation regulatory sequence; and
wherein the ZntA contains an approximately 100 amino acid residue N-terminal extension domain, a first transmembrane spanning domain, a second transmembrane spanning domain containing a putative cation channel motif CPX domain, a third transmembrane spanning domain, a first cytoplasmic domain, a second cytoplasmic domain, and a C-terminal domain.

20. A transgenic plant, or parts thereof, each transformed with a recombinant vector of claim 19.

21. The transgenic plant, or parts thereof according to claim 20, wherein the heavy metal is at least one selected from the group consisting of arsenic, antimony, lead, mercury, cadmium, chromium, tin, zinc, barium, nickel, bismuth, cobalt, manganese, iron, copper, and vanadium.

22. A transgenic plant cell, transformed with a recombinant vector of claim 19.

23. The transgenic plant cell according to claim 22, wherein the heavy metal is at least one selected from the group consisting of arsenic, antimony, lead, mercury, cadmium, chromium, tin, zinc, barium, nickel, bismuth, cobalt, manganese, iron, copper, and vanadium.

24. A transgenic plant, stably transformed with a recombinant vector of claim 19.

25. The transgenic plant according to claim 24, wherein the heavy metal is at least one selected from the group consisting of arsenic, antimony, lead, mercury, cadmium, chromium, tin, zinc, barium, nickel, bismuth, cobalt, manganese, iron, copper, and vanadium.

26. A recombinant vector comprising a coding sequence for a heavy metal-transporting P type ATPase, ZntA
wherein the coding sequence is operably linked to and under the regulatory control of a plant-expressible transcription and translation regulatory;
wherein the ZntA contains an approximately 100 amino acid residue N-terminal extension domain, a first transmembrane spanning domain, a second transmembrane spanning domain containing a putative cation channel motif CPX domain, a third transmembrane spanning domain, a first cytoplasmic domain, a second cytoplasmic domain, and a C-terminal domain; and
wherein each of the domains of the coding sequence shares at least about 70% homology with a same domain of SEQ ID NO:1.

27. A transgenic plant, or parts thereof, each transformed with recombinant vector of claim 26.

28. The transgenic plant, or parts thereof according to claims 27, wherein the heavy metal is at least one selected from the group consisting of arsenic, antimony, lead, mercury, cadmium, chromium, tin, zinc, barium, nickel, bismuth, cobalt, manganese, iron, copper, and vanadium.

29. A transgenic plant cell, transformed with a recombinant vector of claim 26.

30. The transgenic plant, or parts thereof according to claim 29, wherein the heavy metal is at least one selected from the group consisting of arsenic, antimony, lead, mercury, cadmium, chromium, tin, zinc, barium, nickel, bismuth, cobalt, manganese, iron, copper, and vanadium.

31. A transgenic plant, stably transformed with a recombinant vector of claim 30.

32. The transgenic plant according to claim 31, wherein the heavy metal is at least one selected from the group consisting of arsenic, antimony, lead, mercury, cadmium, chromium, tin, zinc, barium, nickel, bismuth, cobalt, manganese, iron, copper, and vanadium.

33. A method of producing a transgenic plant with enhanced resistance to heavy metals comprising:
(a) preparing an expression construct comprising a sequence encoding a heavy metal-transporting P type ATPase, operably linked to and under the regulatory control of a plant-expressible transcription and translation regulatory sequence;
(b) preparing a recombinant vector harboring the expression construct; and
(c) introducing the expression construct of the recombinant vector into a plant cell or plant tissue to produce a transgenic plant cell or transgenic plant tissue.

34. The method of producing a transgenic plant according to claim 33, wherein the heavy metal is at least one selected from the group consisting of arsenic, antimony, lead, mercury, cadmium, chromium, tin, zinc, barium, nickel, bismuth, cobalt, manganese, iron, copper, and vanadium.

35. The method of producing a transgenic plant according to claim 33, further comprising the step of: regenerating a transgenic plant from the transgenic plant cell or transgenic plant tissue of step (c).

36. The transgenic plant or parts thereof according to claim 7, wherein the plant is tobacco.


SEQUENCE LISTING (6 sequences)

SEQ ID NO: 1 - DNA, 2199 bp, Escherichia coli
  zntA gene (1)..(2199)
atgtcgactcctgacaatcacggcaagaaagcccctcaatttgctgcgttcaaaccgctaaccacggtacagaacgccaacgactgttgctgcgacggcgcatgttccagcacgccaactctctctgaaaacgtctccggcacccgctatagctggaaagtcagcggcatggactgcgccgcctgtgcgcgcaaggtagaaaatgccgtgcgccagcttgcaggcgtgaatcaggtgcaggtgttgttcgccaccgaaaaactggtggtcgatgccgacaatgacattcgtgcacaagttgaatctgcgctgcaaaaagcaggctattccctgcgcgatgaacaggccgccgaagaaccgcaagcatcacgcctgaaagagaatctgccgctgattacgctaatcgtgatgatggcaatcagctggggtctggagcagttcaatcatccgttcgggcaactggcgtttatcgcgaccacgctggttgggctgtacccgattgctcgtcaggcattacggttgatcaaatccggcagctacttcgccattgaaaccttaatgagcgtagccgctattggtgcactgtttattggcgcaacggctgaagctgcgatggtgttgctgctgtttttgattggtgaacgactggaaggctgggccgccagccgcgcgcgtcagggcgttagcgcgttaatggcgctgaaaccagaaaccgccacgcgcctgcgtaagggtgagcgggaagaggtggcgattaacagcctgcgccctggcgatgtgattgaagtcgccgcaggtgggcgtttgcctgccgacggtaaactgctctcaccgtttgccagttttgatgaaagcgccctgaccggcgaatccattccggtggagcgcgcaacgggcgataaagtccctgctggtgccaccagcgtagaccgtctggtgacgttggaagtgctgtcagaaccgggagccagcgccattgaccggattctgaaactgatcgaagaagccgaagagcgtcgcgctcccattgagcggtttatcgaccgtttcagccgtatctatacgcccgcgattatggccgtcgctctgctggtgacgctggtgccaccgctgctgtttgccgccagctggcaggagtggatttataaagggctgacgctgctgctgattggctgcccgtgtgcgttagttatctcaacgcctgcggcgattacctccgggctggcggcggcagcgcgtcgtggggcgttgattaaaggcggagcggcgctggaacagctgggtcgtgttactcaggtggcgtttgataaaaccggtacgctgaccgtcggtaaaccgcgcgttaccgcgattcatccggcaacgggtattagtgaatctgaactgctgacactggcggcggcggtcgagcaaggcgcgacgcatccactggcgcaagccatcgtacgcgaagcacaggttgctgaactcgccattcccaccgccgaatcacagcgggcgctggtcgggtctggcattgaagcgcaggttaacggtgagcgcgtattgatttgcgctgccgggaaacatcccgctgatgcatttactggtttaattaacgaactggaaagcgccgggcaaacggtagtgctggtagtacgtaacgatgacgtgcttggtgtcattgcgttacaggataccctgcgcgccgatgctgcaactgccatcagtgaactgaacgcgctgggcgtcaaaggggtgatcctcaccggcgataatccacgcgcagcggcggcaattgccggggagctggggctggagtttaaagcgggcctgttgccggaagataaagtcaaagcggtgaccgagctgaatcaacatgcgccgctggcgatggtcggtgacggtattaacgacgcgccagcgatgaaagctgccgccatcgggattgcaatgggtagcggcacagacgtggcgctggaaaccgccgacgcagcattaacccataaccacctgcgcggcctggtgcaaatgattgaactggcacgcgccactcacgccaatatccgccagaacatcactattgcgctggggctgaaagggatcttcctcgtcaccacgctgttagggatgaccgggttgtggctggcagtgctggcagatacgggggcgacggtgctggtgacagcgaatgcgttaagattgttgcgcaggagataa

SEQ ID NO: 2 - protein, 732 aa, Escherichia coli
  ZntA protein (1)..(732)
MSTPDNHGKKAPQFAAFKPLTTVQNANDCCCDGACSSTPTLSENVSGTRYSWKVSGMDCAACARKVENAVRQLAGVNQVQVLFATEKLVVDADNDIRAQVESALQKAGYSLRDEQAAEEPQASRLKENLPLITLIVMMAISWGLEQFNHPFGQLAFIATTLVGLYPIARQALRLIKSGSYFAIETLMSVAAIGALFIGATAEAAMVLLLFLIGERLEGWAASRARQGVSALMALKPETATRLRKGEREEVAINSLRPGDVIEVAAGGRLPADGKLLSPFASFDESALTGESIPVERATGDKVPAGATSVDRLVTLEVLSEPGASAIDRILKLIEEAEERRAPIERFIDRFSRIYTPAIMAVALLVTLVPPLLFAASWQEWIYKGLTLLLIGCPCALVISTPAAITSGLAAAARRGALIKGGAALEQLGRVTQVAFDKTGTLTVGKPRVTAIHPATGISESELLTLAAAVEQGATHPLAQAIVREAQVAELAIPTAESQRALVGSGIEAQVNGERVLICAAGKHPADAFTGLINELESAGQTVVLVVRNDDVLGVIALQDTLRADAATAISELNALGVKGVILTGDNPRAAAAIAGELGLEFKAGLLPEDKVKAVTELNQHAPLAMVGDGINDAPAMKAAAIGIAMGSGTDVALETADAALTHNHLRGLVQMIELARATHANIRQNITIALGLKGIFLVTTLLGMTGLWLAVLADTGATVLVTANALRLLRRR

SEQ ID NO: 3 - DNA, 42 bp, Artificial Sequence
  Primer
ggatccaaagagtaaagaagaacaatgtcgactcctgacaat

SEQ ID NO: 4 - DNA, 25 bp, Artificial Sequence
  Primer
ggatccctctcctgcgcaacaatct

SEQ ID NO: 5 - DNA, 18 bp, Artificial Sequence
  Primer
gagatgtcgagtctcgaa

SEQ ID NO: 6 - DNA, 23 bp, Artificial Sequence
  Primer
ctcgagcacagtgtagtgactgg